(19) United States
(12) Patent Application Publication
(10) Pub. No.: US 2018/0263828 A1
(43) Pub. Date: Sep. 20, 2018

(54) MULTI-COMPARTMENT PACKAGE FOR FEMININE HYGIENE PRODUCTS

(71) Applicants: Regina Harper, East Point, GA (US); Terance Harper, Atlanta, GA (US)

(72) Inventors: Regina Harper, East Point, GA (US); Terance Harper, Atlanta, GA (US)

(21) Appl. No.: 15/461,711

(22) Filed: Mar. 17, 2017

(51) Int. Cl. A61F 13/551; B65D 65/14; B65D 65/38; B65D 75/58

(52) U.S. Cl. CPC A61F 13/5519; A61F 13/55145; A61F 13/5514; B65D 2575/586; B65D 65/38; B65D 75/5855; B65D 2565/384; B65D 65/14

(57) ABSTRACT

A multi-compartment package (MCP) for feminine hygiene products is disclosed. The MCP may be configured to provide both a feminine hygiene pad and a sanitary wipe in the same package. The MCP may be opened to reveal respective internal compartments for the feminine hygiene pad and a sanitary wipe. These compartments may be separated by way of a compartment divider. Such a configuration may keep a feminine hygiene pad in a desired dry state while still providing a moistened sanitary wipe. An MCP may alleviate the necessity of carrying separate containers for each type of product, instead providing both in a single form factor.

### BACKGROUND

The present disclosure generally relates to feminine hygiene product packaging.

### Background

The menstrual cycle can be a difficult time of the month for women and girls across the globe. Though there are many female hygiene products available to assist during this time (e.g. sanitary wipes and pads), conventional products are separated by type and packaging.

Feminine sanitary wipes are commonly packaged in a plastic pouch or single container with multiple wipes, while feminine hygiene pads are typically individually packaged. With sanitary wipes being external from the pad packaging, this can compel women to carry both types of product, taking up additional space in purses, pockets or bags.

A product that combined both products into a single form factor package could provide convenience and space savings, and alleviate the need to remember to carry both types of product separately.

Therefore, there is a need for an improved multi-compartment package for feminine hygiene products.

### BRIEF OVERVIEW

A multi-compartment package for feminine hygiene products may be provided. This brief overview is provided to introduce a selection of concepts in a simplified form that are further described below in the Detailed Description. This brief overview is not intended to identify key features or essential features of the claimed subject matter. Nor is this brief overview intended to be used to limit the claimed subject matter's scope.

The present disclosure describes packaging that can combine two separate compartments into one individual package, holding one or multiple feminine sanitary wipes and one feminine hygiene pad. The package can include, for example, a package exterior, an interior compartment containing a dry feminine hygiene pad, another interior compartment containing one or more sanitary wipes, and a divider or barrier between the compartments that can keep moisture from the one or more wipes from contacting the dry pad.

A user may utilize the package by opening the wipe compartment (by e.g. pulling a tab) to remove the one or more sanitary wipes, and then opening the pad compartment to remove the feminine hygiene pad.

The package can be disposable. Upon removing the sanitary wipe(s) from the wipe compartment and the feminine hygiene pad(s) from the pad compartment, the user may then discard the empty package.

The packaging, including compartments and interior divider, can comprise metallic (e.g. aluminum), polymeric (e.g. LDPE), paper-based, etc. materials, and may be coated or uncoated. The packaging may include pull tabs, adhesive, self-adhesive materials, crimped or tearable features, etc. to facilitate ease of opening.

Both the foregoing brief overview and the following detailed description provide examples and are explanatory only. Accordingly, the foregoing brief overview and the following detailed description should not be considered to be restrictive. Further, features or variations may be provided in addition to those set forth herein. For example, embodiments may be directed to various feature combinations and sub-combinations described in the detailed description.

### BRIEF DESCRIPTION OF THE DRAWINGS

The accompanying drawings, which are incorporated in and constitute a part of this disclosure, illustrate various embodiments of the present disclosure. The drawings are merely illustrative of various embodiments, elements, and aspects of the present disclosure, and are not intended to reflect scale, depth, dimension, orientation, ordering, thickness, or shape in any limiting way, and are not to be construed to limit the scope of patent protection afforded in any claim of a patent issuing here from, which scope is to be defined by the claims and the equivalents thereof.

The drawings contain representations of various trademarks and copyrights owned by the Applicants. In addition, the drawings may contain other marks owned by third parties and are being used for illustrative purposes only. All rights to various trademarks and copyrights represented herein, except those belonging to their respective owners, are vested in and the property of the Applicant. The Applicant retains and reserves all rights in its trademarks and copyrights included herein, and grants permission to reproduce the material only in connection with reproduction of the granted patent and for no other purpose.

Furthermore, the drawings may contain text or captions that may explain certain embodiments of the present disclosure. This text is included for illustrative, non-limiting, explanatory purposes of certain embodiments detailed in the present disclosure. In the drawings:

FIG. 1A illustrates a top view of a multi-compartment package for feminine hygiene products in accordance with various embodiments.

FIG. 1B illustrates another top view of a multi-compartment package for feminine hygiene products in accordance with various embodiments.

FIG. 1C illustrates a side sectional view of a multi-compartment package for feminine hygiene products in accordance with various embodiments.

FIG. 1D depicts a perspective view of an illustrative open package configuration of a multi-compartment package for feminine hygiene products in accordance with various embodiments.

### DETAILED DESCRIPTION

I. Preliminary Statements

As a preliminary matter, it will readily be understood by one having ordinary skill in the relevant art that the present disclosure has broad utility and application. As should be understood, any embodiment may incorporate only one or a plurality of the above-disclosed aspects of the disclosure and may further incorporate only one or a plurality of the above-disclosed features. Furthermore, any embodiment discussed and identified as being “preferred” is considered to be part of a best mode contemplated for carrying out the embodiments of the present disclosure. Other embodiments also may be discussed for additional illustrative purposes in providing a full and enabling disclosure. Moreover, many embodiments, such as adaptations, variations, modifications, and equivalent arrangements, will be implicitly disclosed by the embodiments described herein and fall within the scope of the present disclosure. Any use of the term “exemplary” (including with regards to an “exemplary embodiment”) is intended to mean, and is to be construed as meaning, “illustrative” or “serving as an example.”

Accordingly, while embodiments are described herein in detail in relation to one or more embodiments, it is to be understood that this disclosure is illustrative and exemplary of the present disclosure, and are made merely for the purposes of providing a full and enabling disclosure. The detailed disclosure herein of one or more embodiments is not intended, nor is to be construed, to limit the scope of patent protection afforded in any claim of a patent issuing here from, which scope is to be defined by the claims and the equivalents thereof. It is not intended that the scope of patent protection be defined by reading into any claim a limitation found herein that does not explicitly appear in the claim itself

Thus, for example, any sequence(s) and/or temporal order of steps of various processes or methods that are described herein are illustrative and not restrictive. Accordingly, it should be understood that, although steps of various processes or methods may be shown and described as being in a sequence or temporal order, the steps of any such processes or methods are not limited to being carried out in any particular sequence or order, absent an indication otherwise. Indeed, the steps in such processes or methods generally may be carried out in various different sequences and orders while still falling within the scope of the present invention. Accordingly, it is intended that the scope of patent protection is to be defined by the issued claim(s) rather than the description set forth herein.

Additionally, it is important to note that each term used herein refers to that which an ordinary artisan would understand such term to mean based on the contextual use of such term herein. To the extent that the meaning of a term used herein—as understood by the ordinary artisan based on the contextual use of such term—differs in any way from any particular dictionary definition of such term, it is intended that the meaning of the term as understood by the ordinary artisan should prevail.

Regarding applicability of 35 U.S.C. § 112, ¶6, no claim element is intended to be read in accordance with this statutory provision unless the explicit phrase “means for” or “step for” is actually used in such claim element, whereupon this statutory provision is intended to apply in the interpretation of such claim element.

Furthermore, it is important to note that, as used herein, “a” and “an” each generally denotes “at least one,” but does not exclude a plurality unless the contextual use dictates otherwise. When used herein to join a list of items, “or” denotes “at least one of the items,” but does not exclude a plurality of items of the list.

The following detailed description refers to the accompanying drawings. Wherever possible, the same reference numbers are used in the drawings and the following description to refer to the same or similar elements. While many embodiments of the disclosure may be described, modifications, adaptations, and other implementations are possible. For example, substitutions, additions, or modifications may be made to the elements illustrated in the drawings, and the methods described herein may be modified by substituting, reordering, or adding stages to the disclosed methods. Accordingly, the following detailed description does not limit the disclosure. Instead, the proper scope of the disclosure is defined by the appended claims. The present disclosure contains headers. It should be understood that these headers are used as references and are not to be construed as limiting upon the subjected matter disclosed under the header.

The present disclosure includes many aspects and features. Moreover, while many aspects and features relate to, and are described in, the context of a multi-compartment package for feminine hygiene products, embodiments of the present disclosure are not limited to use only in this context.

II. Overview

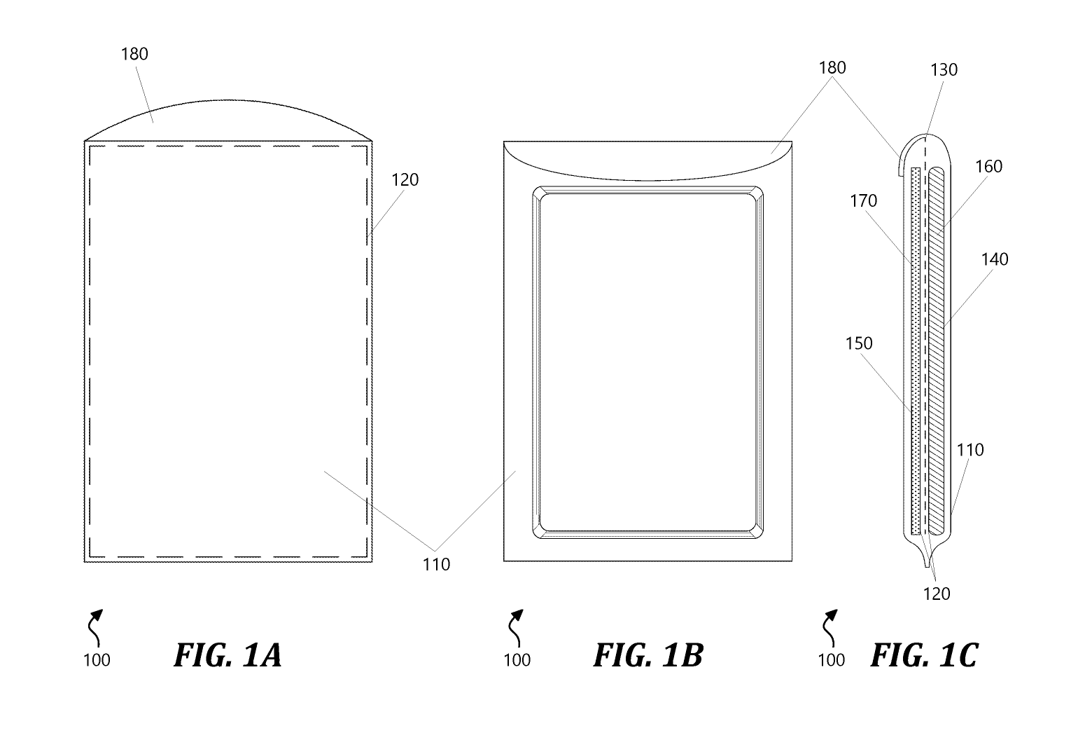

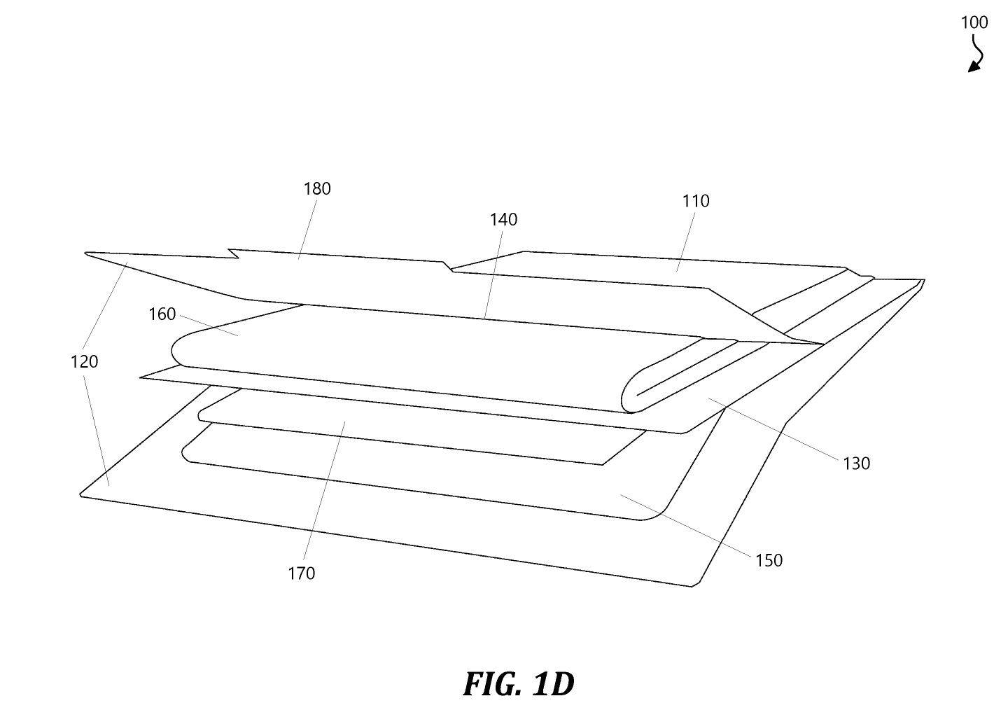

Consistent with embodiments of the present disclosure, a multi-compartment package (MCP) for feminine hygiene products 100 may be provided, as illustrated in FIGS. 1A-D. This overview is provided to introduce a selection of concepts in a simplified form that are further described below. This overview is not intended to identify key features or essential features of the claimed subject matter. Nor is this overview intended to be used to limit the claimed subject matter's scope. A multi-compartment package for feminine hygiene products 100 may be used to provide a convenient single package containing both a sanitary wipe 170 and a feminine hygiene pad 160.

An MCP 100 can have a package exterior 110 and an opening tab 180. The faces of a package exterior 110 can be fixed together—to each other, to an internal compartment divider 130, or both—by a mating element 120. An MCP 100 can have internal compartments, including a wipe compartment 150 and a pad compartment 140. These compartments can contain, respectively, one or more sanitary wipes 170 and one or more feminine hygiene pads 160.

In an exemplary embodiment, a user may open the MCP 100 by way of the opening tab 180, thereby revealing the contents of compartments 140 and 150. The mating element 120 may comprise a resealing mechanism that allows a user to then close the MCP 100.

Various embodiments may be sized and configured so as to fit inside a garment pocket (e.g. a pants or coat pocket), purse, clutch, etc. Such an MCP 100 may provide a conveniently carryable and unobtrusive container for both feminine hygiene pads 160 and sanitary wipes 170.

An MCP 100 in some embodiments can be described as “disposable,” “one-time use,” or “non-reusable,” in that it can carry, for example, a single feminine hygiene pad 160 and single sanitary wipe 170, the provision (for removal) of which is the terminal purpose of the packaging. Upon removing the constituent articles, a disposable MCP 100 may be discarded into a trash receptacle, or if the MCP 100 is recyclable, into a recycling receptacle.

Various embodiments may be partially or wholly recyclable. An MCP 100 that is recyclable may comprise materials that are either themselves recyclable or are immaterial to overall recyclability. For example, a recyclable MCP 100 may have a package exterior 110, opening tab 180, and compartments 140 and 160 made of recyclable papers or plastics, with the inclusion of some adhesive as the mating element 120 not making a material different to the recyclability of the MCP 120.

The universe of possible constituent materials of various MCP 100 components may encompass various polymeric, metallic, and natural fiber materials. Some possible materials include, but are not limited to, polyethylene, polyethylene terephthalate, polyester film (e.g. DuPont™ Mylar®), polypropylene, polystyrene, ionomer resin (e.g. DuPont™ Surlyn®), coated and uncoated papers, woven and nonwoven fabrics, and sheet aluminum.

One version of the MCP 100 may be called a “Fresh'n Pad™”, as it can provide the freshness of a sanitary wipe 170 along with a feminine hygiene pad 160.

III. Configuration

FIGS. 1A-B illustrate a multi-compartment package for feminine hygiene products 100 consistent with embodiments of the present disclosure. The MCP 100 may, but need not necessarily, have rectangular shape when viewed “from above” (i.e. viewing a face rather than an edge).

One possible configuration of the MCP 100 has an opening tab 180 along an edge of the MCP 100, on the package exterior 110. FIGS. 1A-B show an opening tab 180 along the “short” edge of the MCP 100, although an opening tab 180 may be arranged along the “long” edge, along multiple edges, on one or both faces of the MCP 100, or in many other suitable configurations that allow a user to open the MCP 100. An MCP 100 may have may have two or more tabs 180.

A tab 180 may be configured in the “fold-over” style illustrated in FIGS. 1A-B. Alternatively, or in addition, a tab 180 may be a strip, a resealable zipper, a pull tab or string, a peelable adhesive sticker, or another mechanism that facilitates the opening of an MCP 100 by a user. A tab 180 may also comprise a pull-open design (akin to the opening mechanism of a chip bag).

The dotted line illustrates an exemplary placement of mating element 120. A mating element 120 can be a joining, connecting, fastening or sealing mechanism that mates the faces of the MCP 100 to each other. Alternatively, or in addition, mating element 120 can mate one or more faces or edges to each other or a compartment divider 130. Mating element 120 may be a contiguous “surface” (e.g., tracing the perimeter of the MCP 100 as is illustrated in FIGS. 1A-B) or may comprise one or more points, lines or surfaces.

A mating element 120 can comprise an adhesive material. For example, mating element 120 may comprise an “easy release” adhesive that facilitates ease of opening for a user. A mating elements 120 may comprise any suitable adhesive. Such an adhesive may be characterized by a wide range of descriptors and qualities, such as pressure-sensitive, resealable, non-resealable (i.e. one-time use), flexible, multi-part, strip-based, elastomeric, water-resistant, etc.

A mating element 120 may comprise mating mechanisms other than adhesive, such as thermal bonding, mechanical joining or fastening, interlocking protrusions or channels, tearable or separable components, and others. A mating element 120 may comprise a combination of different types of mating mechanisms. For example, a suitable configuration might combine corner “glue dots” with mechanically separable edges.

The package exterior 110 may contain or incorporate one or more indentations. In an embodiment, there can be an indentation on one or both of the “short” (or otherwise opening-parallel) ends. In an embodiment, the package exterior 110 can have a bulge to accommodate the interior package contents.

FIG. 1C illustrates a side sectional view of an MCP 100 consistent with embodiments of the present disclosure. The MCP 100 can have a pad compartment 140 and wipe compartment 150 separated by a compartment divider 130.

A pad compartment 140 may contain, or be configured to contain, one or more feminine hygiene pads 160. Pads 160 may be arranged in a pad compartment 140 in any suitable configuration, including an unfolded, bi-fold, tri-fold, C-fold, Z-fold, gate fold, etc. arrangement.

A wipe compartment 150 may contain, or be configured to contain, one or more sanitary wipes 170. Wipes 170 may be arranged in wipe compartment 150 in any suitable configuration. Wipes 170 may be moistened with various suitable liquid formulations, and may comprise any suitable material, including woven and non-woven materials comprising natural and/or synthetic fibers.

A compartment divider 130 can provide a barrier that separates any liquid or moisture from a sanitary wipe 170 in a wipe compartment 150 from a feminine hygiene pad 160 in a pad compartment 140. As a feminine hygiene pad 160 is designed to be absorbent, it could be disadvantageous for a sanitary wipe 170 or any moisture therefrom to come into contact with the feminine hygiene pad 160.

A compartment divider 130 may be flexible or rigid, and may be designed so as to resist or retain changes in shape (such as folding by a user of the MCP 100 into a desired configuration). In an embodiment, compartment divider 130 can comprise an aluminum sheet. In another embodiment, compartment divider 130 can comprise a textile-based or polymer material. In yet another embodiment, a plastic sheet can be applied or adhesed to compartment divider 130.

Various interior surfaces, such as on the compartment divider 130, pad compartment 140, or wipe compartment 150 can include a surface coating. A surface coating may provide various benefits, such as waterproof/water-resistant properties (“water” here also encompassing any type of liquid that may be present on or in a sanitary wipe 170), or reduced friction for ease of removal of compartment contents. In some embodiments, variously, compartment divider 130, pad compartment 140, wipe compartment 150, or respective surfaces thereof, may comprise or be coated with an ionomer resin such as DuPont™ Surlyn®.

A package exterior 110 may comprise a single type of material, such as plastic or paper. Alternatively, various parts of a package exterior 110 may comprise different materials. Package exterior 110 may be a component distinct from compartments 140 and 150, or may form both the exterior boundary and the interior boundaries of either or both of compartments 140 and 150. More generally any of package exterior 110, mating element 120, compartment divider 130, pad compartment 140, wipe compartment 150, and opening tab 180 may be a part of a contiguous or monolithic component. Likewise, any of the above listed features may (in any combination) be arranged upon, comprise, contain, or be contained by, any of the other above listed features.

Referring now to FIG. 1D, there is shown a conceptual illustration of an exemplary multi-compartment package for feminine hygiene products 100 in an open configuration. In an embodiment consistent with this conceptual illustration, a user may open an MCP 100 by way of an opening tab 180, separating faces of the package exterior 110 that had been joined together by mating element 120, and thereby gaining access to the interior contents of both the pad compartment 140 (namely, a feminine hygiene pad 160) and the wipe compartment 150 (namely, a sanitary wipe 170). Compartments 140 and 150 are depicted as being separated by a compartment divider 130 comprising a thin sheet.

IV. Claims

While the specification includes examples, the disclosure's scope is indicated by the following claims. Furthermore, while the specification has been described in language specific to structural features and/or methodological acts, the claims are not limited to the features or acts described above. Rather, the specific features and acts described above are disclosed as example for embodiments of the disclosure.

Insofar as the description above and the accompanying drawing disclose any additional subject matter that is not within the scope of the claims below, the disclosures are not dedicated to the public and the right to file one or more applications to claims such additional disclosures is reserved.

Although very narrow claims are presented herein, it should be recognized the scope of this disclosure is much broader than presented by the claims. It is intended that broader claims will be submitted in an application that claims the benefit of priority from this application.

The following is claimed:

1. A multi-compartment package for feminine hygiene products comprising:
a wipe compartment;
a pad compartment;
a compartment divider;
one or more mating elements; and
an opening tab.

2. The multi-compartment package for feminine hygiene products of claim 1, wherein the compartment divider comprises an aluminum sheet.

3. The multi-compartment package for feminine hygiene products of claim 1, wherein the one or more mating elements comprise an easy release adhesive material.

4. The multi-compartment package for feminine hygiene products of claim 1, wherein at least one of the one or more mating elements is resealable.

5. The multi-compartment package for feminine hygiene products of claim 1, further comprising a pull tab.

6. The multi-compartment package for feminine hygiene products of claim 1, further comprising at least one feminine hygiene pad and at least one sanitary wipe.

7. The multi-compartment package for feminine hygiene products of claim 6, wherein the at least one feminine hygiene pad is arranged in a folded configuration within the pad compartment.

8. The multi-compartment package for feminine hygiene products of claim 1, wherein the wipe compartment comprises a material that acts as a barrier to liquids that may be present on or in a sanitary wipe contained in the wipe compartment.

9. The multi-compartment package for feminine hygiene products of claim 1, wherein at least one of the wipe compartment and the pad compartment comprises a polymeric material as at least one of:
the composition of the at least one of the wipe compartment and the pad compartment, and
the composition of an interior surface coating on the at least one of the wipe compartment and the pad compartment.

10. The multi-compartment package for feminine hygiene products of claim 9, further wherein the polymeric material comprises an ionomer resin.

11. The multi-compartment package for feminine hygiene products of claim 1, wherein the multi-compartment package for feminine hygiene products comprises recyclable materials.

12. The multi-compartment package for feminine hygiene products of claim 11, further wherein the multi-compartment package for feminine hygiene products is composed entirely of at least one of:
recyclable materials, and
materials that may be included with recyclable materials without impacting the recyclable materials' recyclability.

13. The multi-compartment package for feminine hygiene products of claim 1, wherein the multi-compartment package is dimensioned to fit within a garment pocket.

14. A multi-compartment package system comprising:
a package exterior comprising two faces;
one or more opening tabs;
a wipe compartment;
at least one sanitary wipe contained within the wipe compartment;
a pad compartment;
at least one feminine hygiene pad contained within the pad compartment;
a compartment divider situated between the wipe compartment and the pad compartment; and
one or more mating elements that connect at least one of:
the faces of the package exterior to each other, and
the compartment divider to at least one of the faces of the package exterior,
wherein the one or more opening tabs can be operated to open the multi-compartment package system and expose at least one of the wipe compartment and the pad compartment.

15. The multi-compartment package system of claim 14, wherein the pad compartment comprises an ionomer resin material.

16. The multi-compartment package system of claim 14, wherein the at least one feminine hygiene pad is arranged in a C-fold configuration.

17. The multi-compartment package system of claim 14, wherein the one or more mating elements comprise at least one of:
a resealable adhesive material;
a one-time use adhesive material;
a pressure-sensitive adhesive material;
a zipper mechanism;
a locking mechanism comprising at least one of protrusions, indentations, and channels; and
a mechanically separable interconnecting material.

18. The multi-compartment package system of claim 14, wherein at least one of the one or more opening tabs and at least one of the one or more mating elements are combined into a single element.

19. A feminine hygiene product package comprising:
a waterproof wipe compartment;
one or more sanitary wipes;
a dry pad compartment;
one or more feminine hygiene pads;
a compartment divider separating the waterproof wipe compartment from the dry pad compartment;
an opening tab situated on the exterior of the feminine hygiene product package,
the feminine hygiene product package having an open configuration and a closed configuration, wherein:
the closed configuration is configured so as to keep the one or more sanitary wipes contained in the waterproof compartment and the one or more feminine hygiene pads contained in the dry pad compartment,
further wherein:
the open configuration is configured to allow access to at least one of the one or more sanitary wipes and the one or more feminine hygiene pads.

20. The feminine hygiene product package of claim 19, wherein at least one of the waterproof wipe compartment, the dry pad compartment, and the compartment divider comprises at least one of:
a plastic material;
a paper material;
a textile material;
a metallic material; and
a waterproofing sealant material.
